(19) United States
(12) Patent Application Publication
(10) Pub. No.: US 2005/0214930 A1
(43) Pub. Date: Sep. 29, 2005

(54) Apparatus and method for preventing cross contamination when dissassembling an array

(76) Inventors: Magdalena Anna Bynum, Sunnyvale, CA (US); Dan-Hui Dorothy Yang, Sunnyvale, CA (US); Gary B. Gordon, Saratoga, CA (US); William D. Fisher, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/810,425

(22) Filed: Mar. 26, 2004

(51) Int. Cl. C12M 1/34; C12Q 1/68

(52) U.S. Cl. 435/287.2

(57) ABSTRACT

An array hybridization apparatus and method. The apparatus employs a first substrate that contacts and attaches to an array slide, a second substrate that contacts and attaches to a gasket slide, and a separation means for separating the first substrate from the second substrate. Methods of disassembly of the apparatus are also disclosed.

### FIELD OF THE INVENTION

The invention relates to the field of micro arrays, and more particularly to a novel apparatus and method for preventing cross contamination when disassembling an array. In particular, the invention relates to an apparatus for helping to cleanly separate an array slide from a gasket slide.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on an array slide. These regions (sometimes referenced as “features”) are positioned at respective locations (“addresses”) on the array slide. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, the observed binding pattern will be indicative of the presence and/or concentration of one or more polynucleotide components of the sample.

Biopolymer arrays can be fabricated by depositing previously obtained biopolymers (such as from synthesis or natural sources) onto an array slide, or by in situ synthesis methods. Methods of depositing obtained biopolymers include dispensing droplets to an array slide from dispensers such as pins or capillaries (such as described in U.S. Pat. No. 5,807,522), thermal injets, or pulse jets (such as a piezoelectric inkjet head, as described in PCT publications WO 95/25116 and WO 98/41531, and elsewhere). For in situ fabrication methods, multiple different reagent droplets are deposited stepwise from drop dispensers at a given target location in order to form the final feature (hence a probe of the feature is synthesized on the array stubstrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence is as follows: (a) coupling a selected nucleoside through a phosphite linkage to a functionalized support in the first iteration, or a nucleoside bound to the array slide (i.e. the nucleoside-modified array slide) in subsequent iterations; (b) optionally, but preferably, blocking unreacted hydroxyl groups on the array slide bound nucleoside; (c) oxidizing the phosphite linkage of step (a) to form a phosphate linkage; and (d) removing the protecting group (“deprotection”) from the now array slide bound nucleoside coupled in step (a), to generate a reactive site for the next cycle of these steps. The functionalized support (in the first cycle) or deprotected coupled nucleoside (in subsequent cycles) provides an array slide bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. In both cases, the arrays can be generated in a way that multiple arrays coexist on one slide.

Array slides are typically employed for deposition and in situ arrays. They generally comprise a separate slide with attached or fixed arrays. However, in some cases, the arrays may be deposited and/or attached onto the same slide as the gasket. In other cases a separate gasket slide may be employed.

Gasket slides used for arrays are important because they enclose the polynucleotides used for the hybridizations. A variety of slide materials have been proposed. For instance, the standard slide may comprise a glass slide or similar type material. A typical gasket and/or spacer is then disposed onto the glass, adhered to the glass, or may be pre-cut and attached to the glass. These gasket slides are designed to provide spacing so that the polynucleotides reside in a region defined as a hybridization chamber (in the case of a protein array this would be a binding chamber for protein binding).

The gasket slide and the array slide are most often separated by inserting a wedge between the gasket slide and the array slide. The wedge is then twisted and the gasket slide is separated from the array slide. This technique is problematic since it requires care and manual dexterity so as not to damage the array or lose the solutions held within the gaskets or chambers. In addition, in some cases multiple arrays are manufactured on the same slide to reduce sample size and increase the number of experiments performed on one slide. If multiple samples or different concentrations of the same sample are used, the opening process is very important in order to prevent the sample from one gasket chamber from interacting or contaminating another during the disassembling process. Therefore, there is a substantial need to provide an improved apparatus and method for separation of array slides from gasket slides.

It, therefore, would be desirable to provide an apparatus and method that meets the above described needs and is easy to assemble and disassemble. It would also be desirable to provide an apparatus that is easy to assemble, requires limited parts, and can be automated in separating the gasket slide from the array slide. These and other problems are addressed by the present invention.

### SUMMARY OF THE INVENTION

The invention provides an apparatus for separating an array slide from a gasket slide. The apparatus comprises a first substrate for contacting and attaching to the array slide, a second substrate for contacting and attaching to the gasket slide, and means for separating the first substrate from said second substrate.

The invention also provides a method for disassembling an array hybridization apparatus having a gasket slide contacting an array slide. The method comprises contacting and attaching a first substrate to an array slide, contacting and attaching a second substrate to a gasket slide, and separating the first substrate from the second substrate while also separating the array slide from the gasket slide.

### BRIEF DESCRIPTION OF THE DRAWINGS

Embodiments of the invention will now be described with reference to the drawings, in which:

FIG. 1 illustrates a slide carrying an array, of the present invention;

FIG. 2 is an enlarged view of a portion of FIG. 1 showing ideal spots or features;

FIG. 3 illustrates a slide carrying arrays;

FIG. 4 illustrates a perspective view of the present invention;

FIG. 5 shows a cross sectional view of the present invention;

FIG. 6A shows an enlarged view of a portion of FIG. 4.

FIG. 7A shows a first step in the method of the present invention.

FIG. 7B shows a second step in the method of the present invention.

FIG. 7C shows a third step in the method of the present invention.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “a substrate” includes more than one “substrate”. Reference to a “spacer” or “slide” includes more than one “spacer” or “slide”. In describing and claiming the present invention, the following terminology will be used in accordance with the definitions set out below.

The term “attach” or “to attach” or “attaching” has broad based meaning and comprises various ways for bonding, fastening, reversibly joining, permanently joining, irreversibly joining, adhering, sticking or using an adhesive, placing under vacuum clamping to, fixing, and securing to. For instance, a substrate may “attach” to a gasket or an array slide.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and UNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid reference a liquid containing either a biomonomer or biopolymer, respectively (typically in solution).

A “set” or “sub-set” of any item (for example, a set of features) may contain one or more than one of the item (for example, a set of clamp members may contain one or more such members). An “array”, unless a contrary intention appears, includes any one, two or three dimensional arrangements of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the array slide at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides or polypeptides, are used interchangeably.

The term “adjacent” or “adjacent to” refers to a component or element that is near, next to or adjoining. For instance, a gasket may be adjacent to a spacer.

The term “substantially deformable”, “compressible” or “deformable” shall all have a similar meaning.

The term “slide” refers to any number of materials having at least one planar surface capable of contacting a gasket or spacer. The term shall be broad based to include array slides, polymeric materials, silica based materials, plastics etc. It's important that the “slide” maintain a certain amount of rigidity to compress or deform the gasket and contact the spacer. In certain instances a “slide” will be transparent to allow light to pass through its medium. However, this is not required. The surface may also contain a reflective coating. Also, the “slide” must be capable in certain instances to allow for the mounting or construction of an array or gasket on its surface. Although in certain cases this will not be required if the array is constructed on a separate surface.

All patents and other cited references are incorporated into this application by reference.

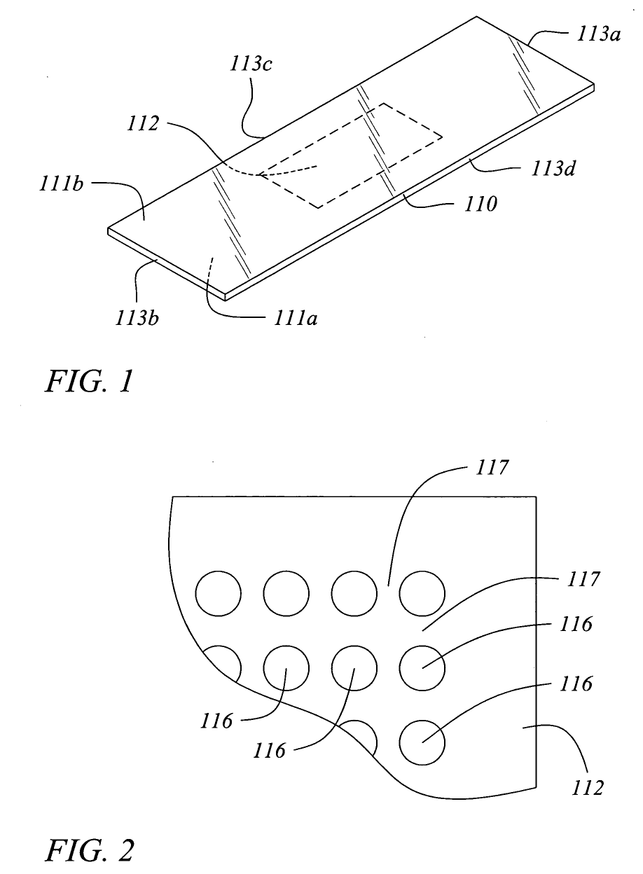

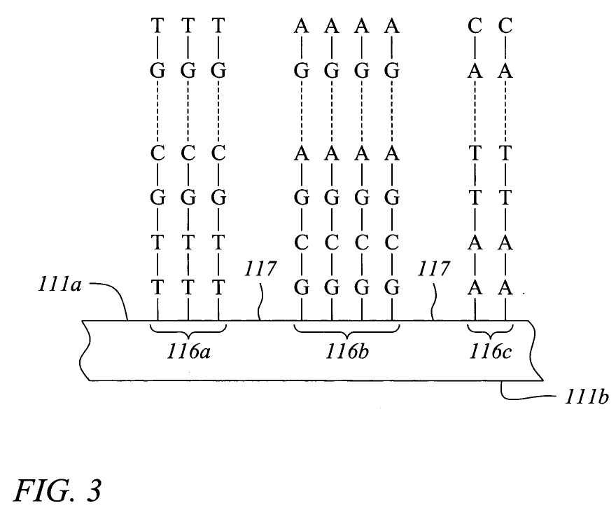

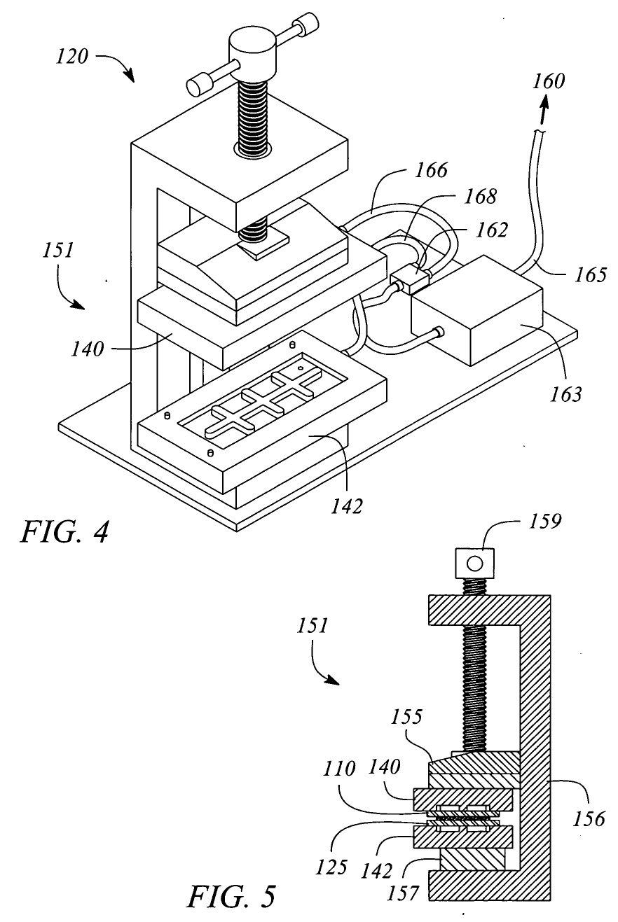

Referring first to FIGS. 1-3, typically the methods and apparatus of the present invention generate or use an array slide 110 carrying an array 112 disposed on a rear surface 111a of an array slide 110. It will be appreciated though, that more than one array (any of which are the same or different) may be present on the rear surface 111a, with or without spacing between such arrays. Note that one or more of the arrays 112 together will cover the entire region of the rear surface 111a, with regions of the rear surface 111a adjacent to the opposed sides 113c, 113d and the leading end 113a and the trailing end 113b of the slide 110. A front surface 111b of the array slide 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown analytes or sequences to be evaluated). The array slide 110 may be of any shape, and any holder used with it adapted accordingly, although the array slide 110 will typically be rectangular in practice. The array 112 contains multiple spots or features 116 of biopolymers in the form of small molecules such as organic drugs, polynucleotides, polypeptides or proteins. A typical array may contain from more than ten, more than one hundred, more than one thousand or ten thousand features, or even more than from one hundred thousand features. All of the features 116 may be different, or some or all could be the same. Features may comprise oligonucleotides and/or proteins/peptides or other biopolymers known in the art. In the case where the array 112 is formed by the conventional in situ or deposition of previously obtained moieties, as described above, by depositing for each feature at least one droplet of reagent such as by using a pulse jet such as an inkjet type head, interfeature areas 117 will typically be present which do not carry any polynucleotide. It will be appreciated though, that the interfeature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide.

The array slide 110 may also carry on the front surface 111b or on the rear side 111a, an identification code in the form of a bar code 115 (not shown in FIGS.) printed on an opaque array slide in the form of a paper label attached by adhesive to the front side 111a. By “opaque” in this context is referenced that the means used to read the bar code 115 (typically a laser beam) can not read the bar code 115 through the label without reading errors. Typically this means that less than 60% or even less than 50%, 30%, 20% or 10% of the signal from the code passes through the array slide. The bar code 115 contains an identification of the array 112 and either contains or is associated with, array layout or layout error information in a manner such as described in U.S. patent applications.

For the purpose of the discussions below, it will be assumed (unless the contrary is indicated) that the array 112 is a polynucleotide or protein array formed by the deposition of previously obtained polynucleotides or proteins using pulse jet deposition units. However, it will be appreciated that an array of other polymers or chemical moieties generally, whether formed by multiple cycles in situ methods adding one or more monomers per cycle, or deposition of previously obtained moieties, or by other methods, may be present instead.

Referring now to FIG. 4, a perspective view of the array hybridization apparatus 120 of the present invention can be seen. The array hybridization apparatus 120 comprises a first substrate 140, a second substrate 142, and a separation means 151. The array hybridization apparatus 120 is designed for receiving a gasket slide 125 attached to an array slide 110 (not shown in FIG. 4). The separation means 151 is designed for separating the first substrate 140 from the second substrate 142. Therefore, if the first substrate 140 attaches to the array slide 110 and the second substrate 142 attaches to the gasket slide 125, then when the separation means 151 separates the first substrate 140 from the second substrate 142 it also separates the gasket slide 125 from the array slide 110. The first substrate 140 and the second substrate 142 may attach to the respective gasket slide 125 and the array slide 110 by a variety of methods known in the art. For instance, attachment may be by way of bonding, adhesive, vacuum, fixing, clipping, and other methods known in the art.

The separation means 151 may comprise a vice having a vice screw and handle 159, vise support 156 and jaws 155 and 157. The first substrate 140 and the second substrate 142 may be mounted or attached to the opposing jaws 155 and 157 respectively. The vice 151 may be opened and closed by turning the vice screw and handle 159.

The first substrate 140 may be mounted or attached to the jaw 155 of the vise 151. The first substrate 140 is designed for contacting and attaching to an array slide 110. The first substrate 140 may comprise a first aperture 170 that may communicate with a first substrate chamber 180. The first aperture 170 is positioned in one or more of the first substrate chambers 180. The first aperture 170 connects one or more of the first substrate chambers 180 with one or more vacuum tubes 165 operatively connected to a vacuum source 160 (See FIG. 4). The vacuum source 160 can, therefore, create a vacuum in one or more of first substrate vacuum chambers 180 to create a vacuum that attaches the array slide 110 to the first substrate 140. Although the figures show one vacuum source 160 the invention should not be interpreted to be limited to this specific design. Other embodiments with one, two, three or multiple vacuum sources are within the scope of the present invention.

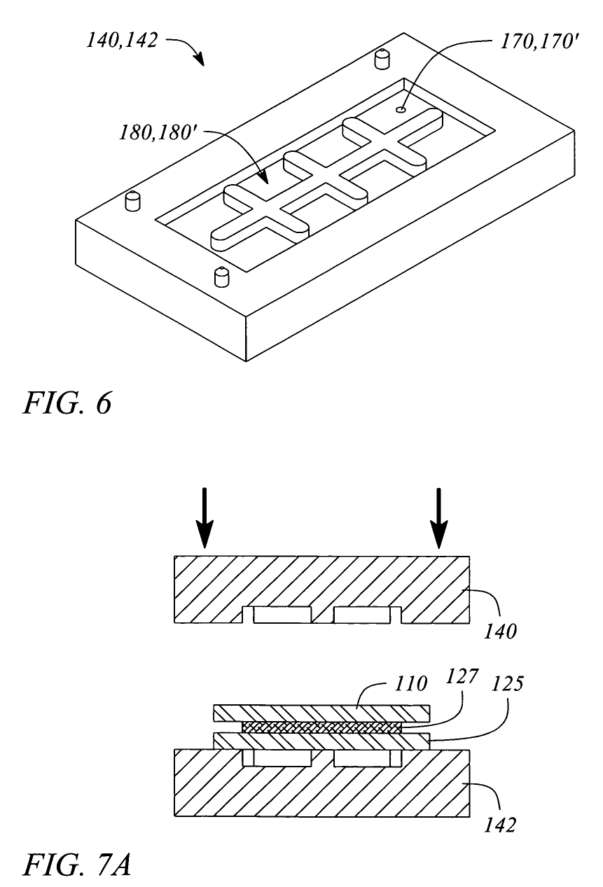

The first substrate chamber 180 may be constructed and/or etched into the first substrate 140. In addition, more than one substrate chamber may be employed and run the length of first substrate 140. For instance, a series of contiguous chambers, i.e. two chambers, four chambers, six chambers, eight chambers, etc. may be positioned across the surface of the first substrate 140 (See FIG. 6). This creates an effective way for creating and evenly distributing a vacuum across an array slide 110 that may be brought in contact with the first substrate 140. A series of ridges are created with separate chambers that evenly distribute a vacuum across the array slide 110 that prevents a strong vacuum from destroying the array slide 110.

The first substrate 140 may comprise a variety of materials. For instance, the materials may comprise wood, metal, plastic, ceramic, composite materials etc. In addition, the first substrate 140 may comprise a variety of shapes and sizes. It is important to the invention that the first substrate 140 comprises a material in which one or more substrate chambers 180 may be etched or constructed in the material.

The second substrate 142 may be mounted or attached to the jaw 157 of the vise 151 (note that the second substrate is not shown since it is similar in design to the first substrate shown in FIG. 6). The second substrate 142 is designed for contacting and attaching to a gasket slide 125. The second substrate 142 may comprise a second aperture 170′ that may communicate with a second substrate chamber 180′. The second aperture 170′ is positioned in one or more of the second substrate chambers 180′. The second aperture 170′ connects one or more of the second substrate chambers 180′ to one or more vacuum tubes that operatively connects to the vacuum source 160. The vacuum source 160 can, therefore, create a vacuum in one or more of second substrate vacuum chambers 180′ to create a vacuum that attaches the gasket slide 125 to the second substrate 142.

The second substrate chamber 180′ may be constructed and/or etched into the second substrate 142. In addition, more than one substrate chamber may be employed and run the length of second substrate 142. For instance, a series of contiguous chambers, i.e. two chambers, four chambers, six chambers, eight chambers, etc. may be positioned across the second substrate 182. This creates an effective way for creating a uniform vacuum across a gasket slide 125 that may be brought in contact with the second substrate 142. A series of ridges are created with separate chambers that evenly distribute a vacuum across the gasket slide 125 that prevents a vacuum from destroying the gasket slide 125.

The second substrate 142 may comprise a variety of materials. For instance, the materials may comprise wood, metal, plastic, ceramic, composite materials etc. In addition, the second substrate 142 may comprise a variety of shapes and sizes. It is important to the invention that the first substrate 142 comprises a material in which one or more substrate chambers 180′ may be etched or constructed in the material.

The separation means 151 may comprise a variety of different devices for separating a gasket slide 125 from an array slide 110. For instance, the separation means 151 may comprise a vice. In addition, the separation means 151 may comprise other devices such as clamps, fasteners, a user's fingers, clips or any other devices known in the art that may exert opposing forces on the gasket slide 125 and the array slide 110.

The vacuum source 160 may comprise any number of sources known in the art. For instance, the vacuum source 160 may comprise a valve connected to a sink that creates a vacuum by flow past an aperture with attached vacuum tubing. The vacuum tubing may then be connected to a vacuum bifurcation valve 162, that splits the vacuum tubing 165 into a first substrate tubing 166 and second substrate tubing 168. The first substrate tubing 166 operatively connects the vacuum source 160 to the first substrate chamber 180 by way of the first aperture 170. The second substrate tubing 168 connects the vacuum source 160 to the second substrate chamber 180′ by way of the second aperture 170′.

After having described the apparatus of the invention, a description of the method of the invention is now in order.

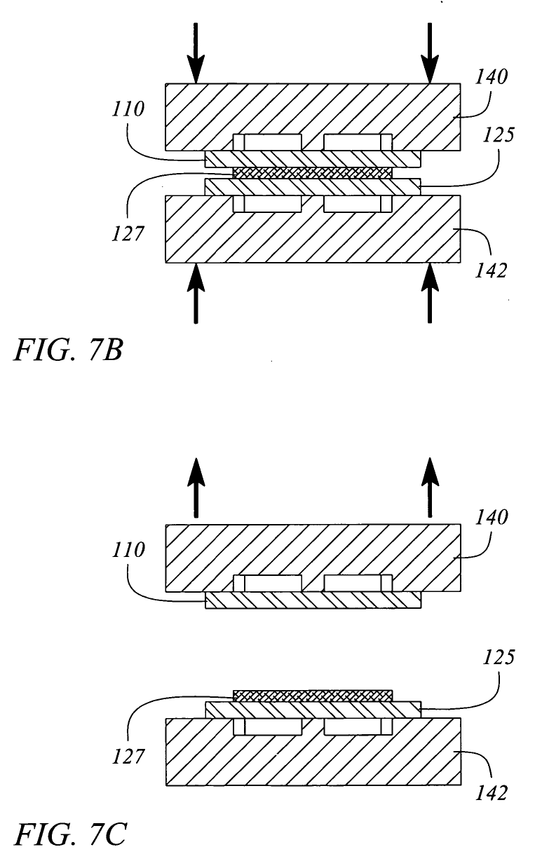

FIGS. 7A-7C show an embodiment of a method of the present invention. The method of the invention begins after the array slide 110 and gasket slide 125 are removed from an array holder (not shown in drawings). The array slide 110 is generally attached or bonded to the gasket slide 125. The gasket slide 125 contains a number of separate array hybridization chambers 131 (not shown if FIGS.) that hold a solution. The gasket slide 125 comprises a gasket 127 and one or more spacers 129 positioned on the gasket slide 125. The array slide 110 is generally positioned opposite the gasket slide 125 and bonds to it by hydrogen bonding, van deer walls forces, ionic bonding, by vacuum or other ways known in the art. It is important to separate the gasket slide 125 from the array slide 110 so as not to spill any of the liquid held in the hybridization chambers 131.

The method of the present invention provides a novel way for separating a gasket slide 125 from an array slide 110. The invention comprises contacting and attaching a first substrate 140 to an array slide 110 (See FIG. 7A), contacting and attaching a second substrate 142 to a gasket slide 125 (See FIG. 7B), and separating the first substrate 140 from the second substrate 142 while also separating the gasket slide 125 from the array slide 110 (See FIG. 7C).

The method of the present invention begins by inserting the joined gasket slide 125 and array slide 110 (See FIG. 7A). The gasket slide 125 that is attached to the array slide 110 is positioned so that the array slide 110 is on top of the gasket slide 125. This positioning is, however, not a requirement of the invention. The slide combination is positioned on the top surface of the second substrate 142.

The second step of the method comprises lowering the first substrate 140 onto the slide combination attached to the second substrate 142 (See FIG. 7B). The first substrate 140 then contacts the slide combination. In particular, the first substrate 140 contacts the array slide 110. The vacuum source 160 is then turned “on” and activated by switch 163. The vacuum is then split between first substrate tubing 166 and second substrate tubing 168. The vacuum or adhesive causes the slide combination to temporarily bond or attach to the second substrate 142.

When the first substrate 140 contacts the array slide 110, the vacuum or adhesive causes the array slide 110 to also attach to the first substrate 140. The first substrate 140 is then separated from the second substrate 142 by way of the separation means 151. This separation also causes the separation of the array slide 110 from the gasket slide 125. When the first substrate is raised or separated from the second substrate the gasket slide 125 is separated from the array slide 110, in an automated fashion so as not to disturb the solutions in gaskets 127. This prevents cross contamination of the samples in various array hybridization chambers 131. Once this final step is completed the array slide 110 or the gasket slide 125 can be removed from the first substrate 140 and/or second substrate 142. It should be noted that although the above method describes particular steps, it is within the scope of the invention that these steps need not occur sequentially or in only the order described. For instance, the vacuum source 160 may be turned “on” before the first substrate 140 is lowered etc. This would allow for the gasket and array slide to bond to the second substrate 142 before any contact is made by the first substrate 140.

Clearly, minor changes may be made in the form and construction of the invention without departing from the scope of the invention defined by the appended claims. It is not, however, desired to confine the invention to the exact form herein shown and described, but it is desired to include all such as properly come within the scope claimed.

1. An apparatus for separating an array slide from a gasket slide, comprising:
(a) a first substrate for contacting and attaching to the array slide;
(b) a second substrate for contacting and attaching to the gasket slide; and
(c) separation means for separating the first substrate from said second substrate.

2. An apparatus as recited in claim 1, wherein the first substrate attaches to the array slide by way of a vacuum.

3. An apparatus as recited in claim 1, wherein the first substrate attaches to the array slide by way of adhesion.

4. An apparatus as recited in claim 1, wherein the first substrate attaches to the array slide by way of bonding to it.

5. An apparatus as recited in claim 1, wherein the second substrate attaches to the gasket slide by way of a vacuum.

6. An apparatus as recited in claim 1, wherein the second substrate attaches to the gasket slide by way of adhesion.

7. An apparatus as recited in claim 1, wherein the second substrate attaches to the gasket slide by way of bonding to it.

8. An apparatus as recited in claim 1, wherein the first substrate is selected from the group consisting of glass, plastic, polymers, thermoplastic materials, metal, wood and composite materials.

9. An apparatus as recited in claim 1, wherein the second substrate is selected from the group consisting of glass, plastic, polymers, thermoplastic materials, metal, wood and composite materials.

10. An apparatus as recited in claim 1, wherein the means for separating the first substrate from the second substrate is selected from the group consisting of a vise, a clamp, a fastener, a machine, a hand, a wedge, and a lever.

11. An apparatus for separating an array slide from a gasket slide, comprising:
(a) a first substrate for contacting the array slide;
(b) a second substrate opposite the first substrate for contacting the gasket slide;
(c) a first vacuum source associated with the first substrate for providing a first vacuum to the first substrate for attaching the first substrate to the array slide; and
(d) a second vacuum source associated with the second substrate for providing a second vacuum to the second substrate for attaching the gasket slide to the second substrate, wherein the first substrate can be separate from the second substrate and the array slide is also separated from the gasket slide.

12. An apparatus for separating an array slide from a gasket slide, comprising:
(a) a first substrate for contacting the array slide;
(b) a second substrate opposite the first substrate for contacting the gasket slide;
(c) a vacuum source associated with the first substrate and second substrate for providing a first vacuum to the first substrate for attaching the first substrate to the array slide and a second vacuum to the second substrate for attaching the gasket slide to the second substrate, wherein the first substrate can be separated from the second substrate and wherein the array slide is also separated from the gasket slide.

13. An apparatus for separating an array slide from a gasket slide, comprising:
(a) a first substrate for contacting the array slide, the first substrate comprising a first means for attaching to the array slide;
(b) a second substrate for contacting and attaching to the gasket slide the second substrate comprising a second means for attaching the second substrate to the gasket slide; and
(c) means for separating said first substrate from said second substrate and concomitantly separating the array slide from the gasket slide.

14. An array hybridization apparatus as recited in claim 1, wherein the gasket comprises a deformable material.

15. An array hybridization apparatus as recited in claim 1, wherein the spacer comprises a substantially non-deformable material.

16. An array hybridization apparatus as recited in claim 1, wherein the gasket is attached to the gasket slide.

17. An array hybridization apparatus as recited in claim 1, wherein the gasket is attached to the array slide.

18. An array hybridization apparatus as recited in claim 1, wherein the gasket comprises a portion of the gasket slide.

19. An array hybridization apparatus as recited in claim 1, wherein the gasket is attached to both the gasket slide and the array slide.

20. An array hybridization apparatus as recited in claim 1, wherein the spacer is attached to the gasket slide.

21. An array hybridization apparatus as recited in claim 1, wherein the spacer is attached to the array slide.

22. An array hybridization apparatus as recited in claim 1, wherein the spacer is attached to both the gasket slide and the array slide.

23. An array hybridization apparatus as recited in claim 1, wherein the spacer comprises a material selected from the group consisting of polyurethanes, polypropylene, plastics, acrylics, metals and non-deformable or less deformable polymers.

24. An array hybridization apparatus as recited in claim 1, wherein the spacer is between 25 to 500 microns in height.

25. An array hybridization apparatus as recited in claim 11, wherein the array hybridization chamber is between 25 to 25,000 microns in height.

26. A method of disassembling an array hybridization apparatus having a gasket slide contacting an array slide, comprising:
a. contacting a first substrate with a vacuum to the array slide;
b. contacting a second substrate with a vacuum to a gasket slide; and
c. separating the first substrate from the second substrate while also separating the gasket slide from the array slide.

27. A method of disassembling an array hybridization apparatus having a gasket slide attached to an array slide, comprising:
a. contacting a second substrate to a gasket slide attached to an array slide;
b. contacting a first substrate to a gasket slide attached to an array slide; and
c. separating the first substrate from the second substrate while also separating the gasket slide from the array slide.
